(19) United States
(12) Patent Application Publication
(10) Pub. No.: US 2026/0305962 A1
(43) Pub. Date: Oct. 8, 2026

(54) A MEDICAL SYSTEM CONFIGURED TO DETERMINE HEALTH CONDITION STATUS BASED ON BLOOD PRESSURE CHANGES DETECTED BY IMPLANTABLE OPTICAL SENSOR

(71) Applicant: Medtronic, Inc., Minneapolis, MN (US)

(72) Inventors: Veronica Ramos, Miami, FL (US); Todd M. Zielinski, McGrath, MN (US); Shantanu Sarkar, Roseville, MN (US); Yong K. Cho, Excelsior, MN (US); Richard J. O'Brien, Hugo, MN (US); Douglas A. Hettrick, White Bear Lake, MN (US)

(21) Appl. No.: 19/478,979

(22) PCT Filed: Apr. 3, 2024
(86) PCT No.: PCT/IB2024/053231

§ 371 Date: Oct. 27, 2025

Related U.S. Application Data
(60) Provisional application No. 63/498,903, filed on Apr. 28, 2023.

(51) Int. Cl. A61B 5/0215; A61B 5/00; A61B 5/0205

(52) U.S. Cl. CPC A61B 5/02158; A61B 5/0205; A61B 5/7267; A61B 5/7275

(57) ABSTRACT

An example implantable medical device includes a housing configured for subcutaneous implantation at a location within a patient; an optical sensor, the optical sensor being at least one of on or within the housing, the optical sensor configured to generate optical signals indicative of blood movement in vessels of the patient proximate to the location over a period of time; and processing circuitry configured to: determine a plurality of blood pressure levels over the period of time based on the optical signals; determine blood pressure changes over the period of time based on the determined plurality of blood pressure levels; determine a health condition status of the patient based on the determined blood pressure changes over the period of time; and output an indication of the determined health condition status.

This application claims the benefit of U.S. Provisional Patent Application Ser. No. 63/498,903, filed Apr. 28, 2023, the entire content of which is incorporated herein by reference.

### TECHNICAL FIELD

This disclosure generally relates to assessing a health condition status based on blood pressure.

### BACKGROUND

Hypertension represents a key factor into the development of cardiovascular disease. 1 out of 3 adults in the United States are diagnosed with hypertension every year. Uncontrolled hypertension dramatically increases risk for maladies, such as coronary artery disease, congestive heart failure, renal failure, eyesight damage and stroke. Consequently, blood pressure measurement and monitoring is a key indicator critical to treatment and management of the overall patient's health.

### SUMMARY

In general, this disclosure is directed to techniques for detecting changes in blood pressure based on one or more signals generated by an optical sensor of an implantable medical device to, for example, facilitate a determination of a status of a health condition of a patient, such as hypertension. More particularly, the disclosure is directed to techniques for evaluating optical signals to determine changes in blood pressure over a period of time, e.g., one or more days, to determine a health condition status of a patient Processing circuitry may extract particular features from the received optical signal and apply the extracted features to an artificial intelligence model, such as a machine learning model or other suitable model, to determine blood pressure changes over the period of time and/or determine a health condition status of the patient.

For many patients, blood pressure is only measured during clinic and/or hospital visits. For such patients, changes in blood pressure and health condition status between visits may go undetected. Some patients may use conventional mechanical plethysmographic blood pressure measurement devices at home, but still with relatively infrequent measurements having a quality dependent on the compliance and competence of the patient or a caregiver.

For some medical devices, such as watches, patches, bands, fitness, trackers, or other external devices configured to sense blood pressure, e.g., using photophethysmograpic techniques, the location and orientation of the sensors used to sense blood pressure values may vary between patients, and within a given patient over time. External medical devices also require the user to comply with wearing the device.

The techniques and systems of this disclosure may be implemented in an implantable medical device (IMD) that can continuously, e.g., autonomously and on a periodic and/or triggered basis, sense optical signals that indicate blood pressure values without human intervention while subcutaneously implanted in a patient over months or years. The continuously monitored optical signals and/or features continuously derived from the continuously monitored optical signals, used by the processing circuitry to determine a health condition status of the patient based on the determined blood pressure changes over the period of time may improve sensitivity and/or specificity of blood pressure changes. For example, by continuously monitoring blood pressure and determining changes in blood pressure in the continuously monitored blood pressure, leads to improved sensitivity and/or specificity of blood pressure changes as these determined blood pressure changes may not be capable of being determined by in-office visits or by a patient manually taking blood pressure measurements themselves. In some examples, improving the sensitivity and/or specificity of blood pressure changes over a period of time may facilitate more accurate determinations of hypertension, cardiac wellness, and risk of sudden cardiac death, and may lead to clinical interventions to suppress hypertension such as medications and ablations.

Unlike conventional blood pressure detection systems, the techniques and systems of this disclosure may use a machine learning model to more accurately determine a health condition status of the patient. In some examples, the machine learning model is trained with a set of training instances, where one or more of the training instances comprise data that indicate relationships between various features of the received optical signal (including features for particular blood pressure levels) and classifications of changes of blood pressure levels over a period of time. Because the machine learning model is trained with potentially thousands or millions of training instances, the machine learning model may reduce the amount of classification error in classifying a health condition status based on blood pressure changes when compared to conventional blood pressure detection systems. Additionally, the techniques and systems of this disclosure may be implemented in an implantable medical device (IMD) that can continuously, e.g., autonomously and on a periodic and/or triggered basis, sense blood pressure values without human intervention while subcutaneously implanted in a patient over months or years. Processing circuitry of the IMD or other devices of a medical system including the IMD may perform millions of operations per second on received optical signal data to determine blood pressure changes to determine a health condition status of the patient, e.g., with a machine learning model. Using techniques of this disclosure with an IMD may be advantageous when a physician cannot be continuously present with the patient over weeks or months to evaluate blood pressure levels and/or where performing millions of operations on weeks or months of blood pressure levels could not practically be performed in the mind of a physician with techniques of this disclosure, e.g., including use of a machine learning model.

Reducing classification errors for a health condition status with a machine learning model implementing techniques of this disclosure may provide one or more technical and clinical advantages. In some examples, a medical system determining a plurality of blood pressure values over a period time based on received optical signals, determining blood pressure changes over the period of time based on the blood pressure levels, and determining a health condition status of the patient based on the determined blood pressure changes over the period of time, for example by applying a trained machine learning model to features extracted from the received optical signals, may help the determination of a health condition status, such as a degree of hypertension, to have higher specificity and sensitivity. For example, when a system or computing device determining a health condition status of a patient, such as a degree of hypertension, has higher specificity and sensitivity, a number of false positives may be reduced. In some examples, using a machine learning model as described in this disclosure that may result in higher specificity and sensitivity of determining a health condition status of a patient, such as a degree of hypertension. This higher specificity and sensitivity may increase reliability of another device, user, and/or clinician on the accuracy of determining a health condition status of a patient, such as a degree of hypertension. In some examples, this improved reliability on the accuracy of determining a health condition status of a patient, such as a degree of hypertension, may result in improved usefulness of the system or computing device as a clinician, user, or other computing device may not use and/or rely upon determinations that are not at or above a specificity and sensitivity threshold. Systems and techniques of this disclosure using a machine learning model may also more flexibly classify or predict a health condition status of a patient, such as a degree of hypertension, from particular portions of optical signals by eliminating the need to configure explicit rule sets in an IMD that may otherwise be too expansive in size to practically implement and process for each new portion of optical signals sensed for a patient. With more accurate health condition status classifications, such as degree of hypertension, provided by the machine learning model used with techniques of this disclosure, physicians and caregivers may also provide better-tailored care, therapies, and interventions for the patient experiencing a health event, such as hypertension.

In one example, this disclosure describes an implantable medical device comprising: a housing configured for subcutaneous implantation at a location within a patient; an optical sensor, the optical sensor being at least one of on or within the housing, the optical sensor configured to generate optical signals indicative of blood movement in vessels of the patient proximate to the location over a period of time; and processing circuitry configured to: determine a plurality of blood pressure levels over the period of time based on the optical signals; determine blood pressure changes over the period of time based on the determined plurality of blood pressure levels; determine a health condition status of the patient based on the determined blood pressure changes over the period of time; and output an indication of the determined health condition status.

In another example, this disclosure describes a system comprising an implantable medical device (IMD) comprising: a housing configured for subcutaneous implantation at a location within a patient; and an optical sensor, the optical sensor being at least one of on or within the housing, the optical sensor configured to generate optical signals indicative of blood movement in vessels of the patient proximate to the location over a period of time; and one or more computing devices communicatively coupled to the IMD, wherein the one or more computing devices comprise: a memory; and processing circuitry coupled to the memory, the processing circuitry being configured to: determine a plurality of blood pressure levels over the period of time based on the optical signals; determine blood pressure changes over the period of time based on the determined plurality of blood pressure levels; determine a health condition status of the patient based on the determined blood pressure changes over the period of time; and output an indication of the determined health condition status.

In another example, this disclosure describes a method for operating processing circuitry of a medical system comprising: receiving, by the processing circuitry, optical signals indicative of blood movement in vessels of a patient via an implantable medical device, including an optical sensor, implanted subcutaneously within the patient; determining, by the processing circuitry, a plurality of blood pressure levels of the patient over a period of time based on the optical signals; determining, by the processing circuitry, blood pressure changes over the period of time based on the determined plurality of blood pressure levels; determining, by the processing circuitry, a health condition status of the patient based on the determined blood pressure changes over the period of time; and outputting, by the processing circuitry, an indication of the determined health condition status.

In another example, this disclosure describes a non-transitory computer-readable storage medium having stored thereon instructions that, when executed, cause one or more processors to at least: receive optical signals indicative of blood movement in vessels of a patient via an implantable medical device, including an optical sensor, implanted subcutaneously within the patient; determine a plurality of blood pressure levels of the patient over a period of time based on the optical signals; determine blood pressure changes over the period of time based on the determined plurality of blood pressure levels; determine a health condition status of the patient based on the determined blood pressure changes over the period of time; and output an indication of the determined health condition status.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1A is a conceptual diagram illustrating an example system for determining a health condition status, in accordance with some examples of the current disclosure.

FIG. 1B is a block diagram illustrating an example system that includes an access point, a network, external computing devices, such as a server, and one or more other computing devices, which may be coupled to the IMD and/or computing device of FIG. 1A.

FIG. 2A is a block diagram illustrating an example configuration of the IMD of FIGS. 1A and 1B, in accordance with some examples of the current disclosure.

FIG. 2B is a block diagram illustrating an example configuration of the computing device of FIG. 1A, in accordance with some examples of the current disclosure.

FIGS. 3A-3C are conceptual diagrams of sensed pulsatile waveforms in accordance with some examples of the current disclosure.

FIG. 4A is a conceptual perspective diagram illustrating an example configuration of the IMD of FIGS. 1A-2A.

FIG. 4B is a schematic diagram illustrating an example configuration of the IMD of FIGS. 1A-2A.

FIG. 4C is a conceptual perspective diagram illustrating an example configuration of the IMD of FIGS. 1A-2A.

FIG. 5 is a flow diagram illustrating an example technique for operating a system to determine a health condition status.

FIG. 6 is a conceptual diagram illustrating an example machine learning model configured to determine a blood pressure level, changes in blood pressure level, and/or a health condition status.

FIG. 7 is a conceptual diagram illustrating an example training process for an artificial intelligence model, in accordance with examples of the current disclosure.

FIGS. 8A-8C are flow diagrams of determining a health condition status based on optical signals in accordance with some examples of the current disclosure.

FIGS. 9A-9B are conceptual diagrams of determining a health condition status based on applying optical signals to a respective machine learning model.

### DETAILED DESCRIPTION

A variety of types of medical devices sense blood pressure of patient. An implantable medical device (IMD) may include an optical sensor to sense blood pressure. The optical sensors used by IMDs to sense blood pressure may be integrated with a housing of the IMD and/or coupled to the IMD via one or more elongated leads. Example IMDs that may be configured to monitor blood pressure include pacemakers and implantable cardioverter-defibrillators, which may be coupled to intravascular or extravascular leads, as well as pacemakers with housings configured for implantation within the heart, which may be leadless. An example of pacemaker configured for intracardiac implantation is the Micra™ Transcatheter Pacing System, available from Medtronic, Incc. Some IMDs that do not provide therapy, e.g., implantable patient monitors, may be configured to sense blood pressure. One example of such an IMD is the Reveal LINQ™ and LINQ II™ Insertable Cardiac Monitors (ICMs), available from Medtronic, Inc., which may be inserted subcutaneously. Such IMDs may facilitate relatively longer-term monitoring of patients during normal daily activities, and may periodically transmit collected data to a network service, such as the Medtronic Carelink™ Network.

Any medical device configured to sense blood pressure values via implanted optical sensors, including the examples identified herein, may implement the techniques of this disclosure for evaluating optical signals to determine a health condition status of a patient based on changes in blood pressure over time. For example, particular features may be extracted from the optical signals, the extracted features may be applied to a machine learning model to determine a health condition status, which lead to accurate determinations of a health condition status, such as a degree of hypertension, with high specificity and sensitivity. The techniques of this disclosure for determining a health condition status of a patient, such as a degree of hypertension, may facilitate determinations of a degree of hypertension, cardiac wellness, and risk of sudden cardiac death, and may lead to clinical interventions to suppress hypertension such as medications and ablations.

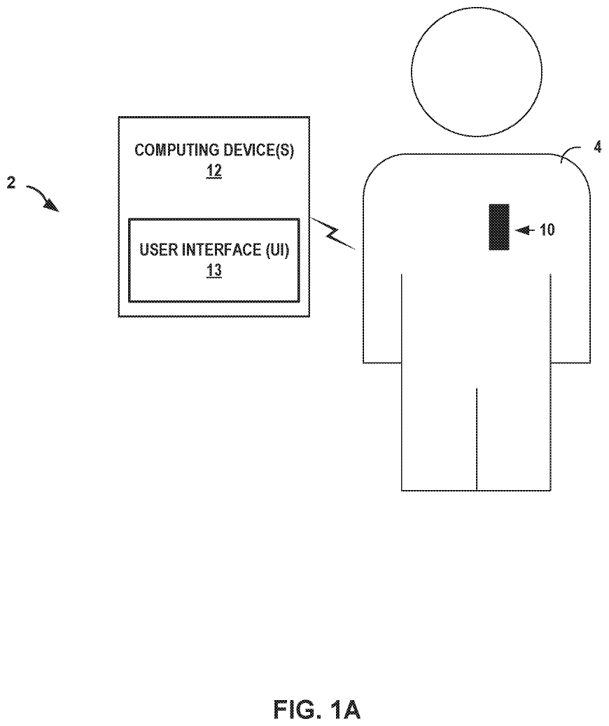

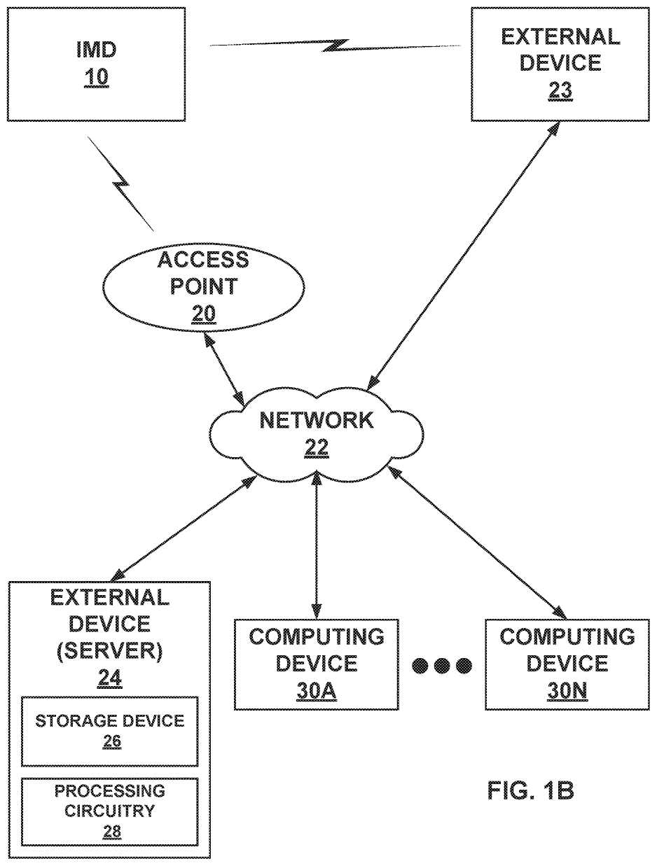

FIG. 1A is a conceptual diagram illustrating an example system 2 for determining a health condition status, using a machine learning model, based on receiving signals from an optical sensor. As shown in FIG. 1A, system 2 includes a computing device 12. Computing device 12 may be a computing device used in a home, ambulatory, clinic, or hospital setting. Computing device 12 may include, for example, a clinician programmer, a desktop computer, a laptop computer, a workstation, a server, a mainframe, a cloud computing system, a smartphone, a smartwatch, combinations thereof, or the like. Computing device 12 may be configured to receive, via a user interface device 13 (“UI 13”), input from a user, such as a clinician or patient 4, output information to a user, or both. In some examples, UI 13 may include a display (e.g., a liquid crystal display (LCD) or light emitting diode (LED) display), such as a touch-sensitive display; one or more buttons; one or more keys (e.g., a keyboard); a mouse; one or more dials; one or more switches; a speaker; one or more lights; combinations thereof; or the like.

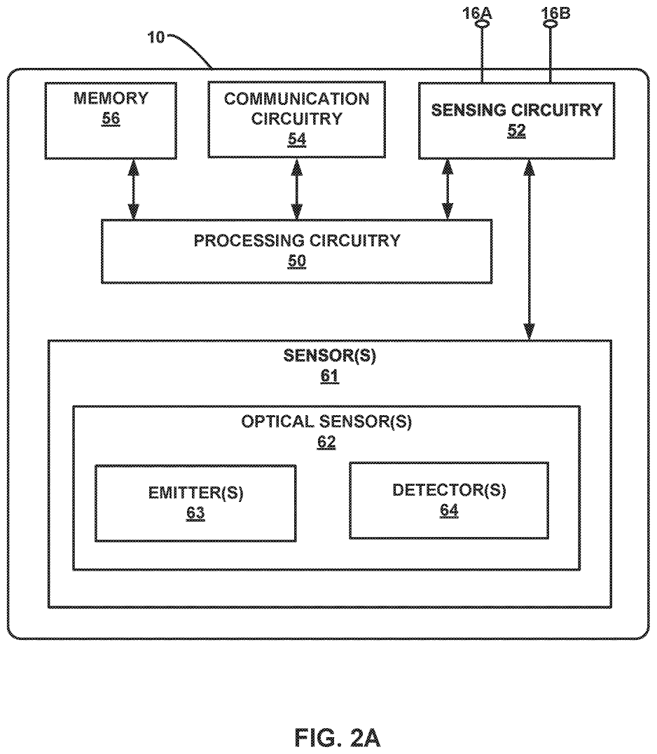

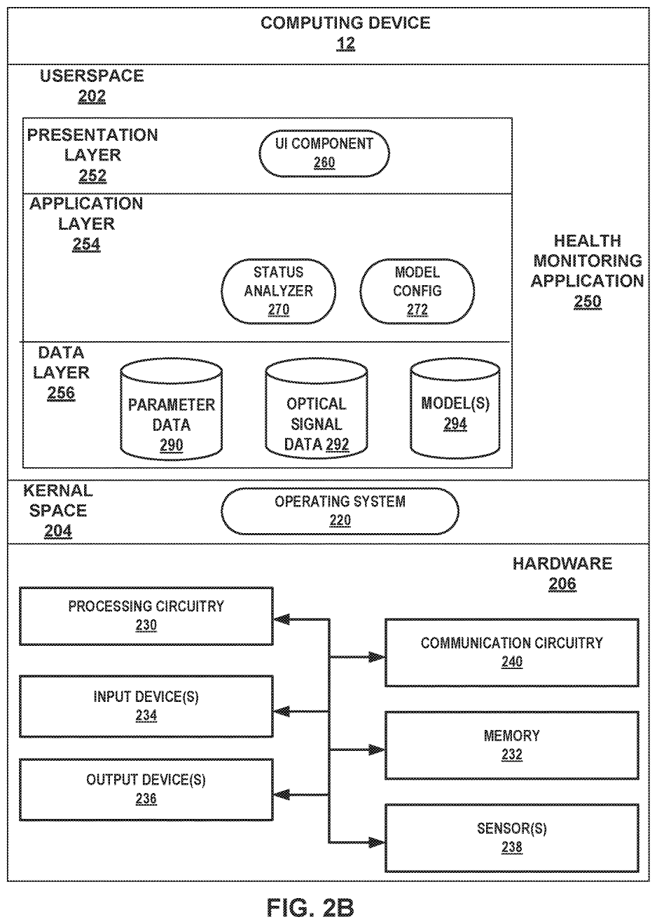

Computing device 12 may be communicatively coupled to an implantable medical device (IMD) 10. In some examples, IMD 10 may be configured to be implanted subcutaneously in patient 4 and may include an optical sensor(s) 62 (as shown in FIG. 2A). In some examples, IMD 10 may include additional sensor(s) 61 (as shown in FIG. 2A) to the optical sensor(s) 62. Some examples of physiological signals that may be sensed by such devices may include blood pressure, electrocardiogram (ECG) signals, heart rate, cardiac output, heart sounds, impedance, cardiac motion, respiration signals, perfusion signals, activity and/or posture signals, pressure signals, blood oxygen saturation signals, body composition, fluid impedance signals, and blood glucose or other blood constituent signals. In some examples, IMD 10 may include electrodes and other sensors 61, in addition to optical sensor(s) 62 to sense physiological signals of patient 4, and may collect and store physiological data and detect episodes based on such signals. In some examples, the optical sensor(s) 62 of IMD 10 is a photoplethysmography (PPG) sensor. In some examples, IMD 10 takes the form of the Reveal LINQ™ or LINQ II ICM™, or another ICM similar to, e.g., a version or modification of, the LINQ™ ICMS.

IMD 10 may be configured to collect and/or communicate the sensed physiological signals and/or data based on the sensed physiological signals to computing device 12. For examples, IMD 10 may detect blood pressure values of patient 4 and collect and/or communicate the detected blood pressure values with computing device 12. In some examples, a subcutaneous optical sensor, such as a PPG sensor, of IMD 10 may be placed at a desired location. The subcutaneous optical sensor signal may reflect blood movement in the vessels, going from the heart to the measuring location, in a wave-like motion. The output of the subcutaneous optical sensor 62 over a period of time may be used as a surrogate for blood pressure levels and blood pressure changes in patient 4.

With every contraction of the left ventricle of the heart, the left ventricle ejects blood to generate a pressure pulse that travels throughout the arteries of the patient. This pulse is detectable at various locations of a patient, including at various subcutaneous implantable positions. The IMD 10 may be configured to be implanted subcutaneously. Optical sensor(s) 62 may be located in IMD 10.

In accordance with techniques of the present disclosure, a IMD 10 and/or computing device 12 may determine a health condition status of a patient based on changes in blood pressure over time, where changes in blood pressure may be changes in various features from a PPG waveform. In some examples, computing device 12 may receive hemodynamic parameter(s) of a patient, such as blood pressure, from IMD 10. In some examples, the hemodynamic parameter may be blood pressure.

FIG. 1B is a block diagram illustrating an example system that includes an access point 20, a network 22, external computing devices, such as a server 24, and one or more other computing devices 30A-30N (collectively, “computing devices 30”), which may be coupled to IMD 10 and external device 23 via network 22, in accordance with one or more techniques described herein. In this example, IMD 10 may use communication circuitry 54 to communicate with external device 23 via a first wireless connection, and to communicate with an access point 20 via a second wireless connection. In the example of FIG. 1B, access point 20, external device 23, server 24, and computing devices 30 are interconnected and may communicate with each other through network 22. In some examples, external device 23 in FIG. 1B may be computing device 12 as shown in FIG. 1A.

Access point 20 may include a device that connects to network 22 via any of a variety of connections, such as telephone dial-up, digital subscriber line (DSL), or cable modem connections. In other examples, access point 20 may be coupled to network 22 through different forms of connections, including wired or wireless connections. In some examples, access point 20 may be a user device, such as a tablet or smartphone, that may be co-located with the patient. IMD 10 may be configured to transmit data, such as optical signals, to access point 20. Access point 20 may then communicate the retrieved data to server 24 via network 22.

In some cases, server 24 may be configured to provide a secure storage site for data that has been collected from IMD 10 and/or external device 23. In some cases, server 24 may assemble data in web pages or other documents for viewing by trained professionals, such as clinicians, via computing devices 30. One or more aspects of the illustrated system of FIG. 1B may be implemented with general network technology and functionality, which may be similar to that provided by the Medtronic CareLink® Network. In some examples, server 24 may communicate with computing device 30 via network 22. For example, server 24 may communicate an analysis of data, such as blood pressure changes, to computing device 30, external device 23, or any other computing device via network 22. For example, server 24 may communicate a health condition status or blood pressure changes to computing device 30, external device 23, or any other computing device via network 22.

In some examples, one or more of computing devices 30 may be a tablet or other smart device located with a clinician, by which the clinician may program, receive alerts from, and/or interrogate IMD 10. For example, the clinician may access data collected by IMD 10 through a computing device 30, such as when patient 4 is in in between clinician visits, to check on a status of a medical condition. In some examples, the clinician may enter instructions for a medical intervention for patient 4 into an application executed by computing device 30, such as based on a status of a patient condition determined by IMD 10, external device 23, server 24, or any combination thereof, or based on other patient data known to the clinician. Device 30 then may transmit the instructions for medical intervention to another of computing devices 30 located with patient 4 or a caregiver of patient 4. For example, such instructions for medical intervention may include an instruction to change a drug dosage, timing, or selection, to schedule a visit with the clinician, or to seek medical attention. In further examples, a computing device 30 may generate an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 1B, server 24 includes a storage device 26, e.g., to store data retrieved from IMD 10, and processing circuitry 28. Although not illustrated in FIG. 1B computing devices 30 may similarly include a storage device and processing circuitry. Processing circuitry 28 may include one or more processors that are configured to implement functionality and/or process instructions for execution within server 24. For example, processing circuitry 28 may be capable of processing instructions stored in memory 26. Processing circuitry 28 may include or be coupled to communication circuitry that may include any suitable hardware, firmware, software or any combination thereof for communicating with another device. In some examples, a description of processing circuitry 28 outputting a signal, such as a classification, may include processing circuitry 28 causing communication circuitry of server 4 to output the signal. Processing circuitry 28 may include, for example, microprocessors, DSPs, ASICs, FPGAs, or equivalent discrete or integrated logic circuitry, or a combination of any of the foregoing devices or circuitry. Accordingly, processing circuitry 28 may include any suitable structure, whether in hardware, software, firmware, or any combination thereof, to perform the functions ascribed herein to processing circuitry 28. Processing circuitry of computing device 12, processing circuitry 28 of server 24 and/or the processing circuity of computing devices 30 may implement any of the techniques described herein to analyze optical signals received from IMD 10, e.g., to determine a health condition status of patient 4. For example, processing circuitry 28 may determine a health condition status of patient 4 based on changes in blood pressure over time, where changes in blood pressure may include changes in various features from a PPG waveform.

Storage device 26 may include a computer-readable storage medium or computer-readable storage device. In some examples, memory 26 includes one or more of a short-term memory or a long-term memory. Storage device 26 may include, for example, RAM, DRAM, SRAM, magnetic discs, optical discs, flash memories, or forms of EPROM or EEPROM. In some examples, storage device 26 is used to store data indicative of instructions for execution by processing circuitry 28.

FIG. 2A is a block diagram illustrating an example configuration of IMD 10 of FIGS. 1A and 1B. As shown in FIG. 2A, IMD 10 may include processing circuitry 50, memory 56, one or more sensor(s) 61, which may include one or more optical sensor(s) 62, sensing circuitry 52 coupled to one or more sensors 61 and electrodes 16A and 16B (collectively “electrodes 16”), and communication circuitry 54. As used herein, “sensors” may refer to any sensors described herein, including electrodes 16 and optical sensors 62.

One or more sensor(s) 61 of IMD 10 may sense physiological parameters or signals of patient 4. Sensor(s) 61 may include one or more accelerometers (e.g., 3-axis accelerometers), temperature sensors, pressure sensors, heart sound sensors (e.g., microphones or accelerometers), or other sensors. Electrodes 16 may be configured to sense cardiac electrograms or other electrogram signals of patient 4, and/or impedance of tissue fluid proximate the electrodes. One or more optical sensor(s) 62 may include one or more light detector(s) 64 configured to receive and/or detect light signals, such as reflected light signals originating from light emitter(s) 63. Light signals received and/or detected by optical sensor(s) 62 may be referred to as optical signals. In some examples, one or more of optical sensor(s) 62 may be configured as a PPG sensor, e.g., by being configured to receive light reflected by blood in one or more blood vessels. In some examples, an optical sensor 62 may be included in a same sensor package and/or may be implemented using the same transducer(s). IMD 10 may, in some cases, include one or more optical sensors 62 including two or more light emitters 63 and one or more light detectors 64.

An IMD 10, e.g., optical sensor 62, may produce a signal proportional to instantaneous blood pressure variations. In some examples, IMD 10 and/or optical sensor 62 may be calibrated such that IMD 10 may be able to determine blood pressure values based on the signal. In some examples in which IMD 10 is not so calibrated, the optical signal may nevertheless be useful for tracking variations in the amplitude/morphology of blood pressure, such as systolic and/or diastolic blood pressure and other features as described herein, over a period of time. In some examples, this period of time may be one week, two weeks, one month, two months, and/or other periods of time. The days used in the period of time may be adjacent days (e.g., such as Monday through Friday) or may be non-adjacent days (e.g., such as every Monday).

Although the techniques for determining a health condition status are described herein as being performed by processing circuitry 50 of IMD 10, such techniques may be performed, in whole or part, by processing circuitry of any one or more devices of system 2, such as processing circuitry 230 of computing device 12, processing circuitry 28 of server 24, or processing circuitry of another computing device or computing system, such as a different implantable medical device, server, cloud computing system, or any other computing device or combination of computing devices.

Processing circuitry 50 may be configured to determine a plurality of blood pressure levels over the period of time based on the optical signals. In some examples, blood pressure levels may include one or more of systolic blood pressure or diastolic blood pressure of patient 4. In some examples, blood pressure levels may be values derived from optical signals at different times. In some examples, optical signals and/or blood pressure levels may indicate a variety of features, such as the cardiac features further described below. Processing circuitry 50 may determine blood pressure changes over the period of time based on the determined plurality of blood pressure levels, determine a health condition status of the patient based on the determined blood pressure changes over the period of time, and output an indication of the determined health condition status. In some examples, the health condition status is a degree of hypertension.

In some examples, processing circuitry 50, using the plurality of blood pressure levels over a period of time, such as three-months, may determine various changes in blood pressure levels during that period of time, including short-term changes (e.g., changes in daily mean, mode, median) and/or long term-changes (e.g., changes in weekly mean, mode, median, monthly mean, mode, median). Based on these various determined changes, processing circuitry 50 may determine a health condition status of the patient, such as an increase/decrease in hypertension, an increase/decrease in hypertension risk score, or other changes. Processing circuitry 50 may output an indication of the determined health condition status to computing device 12 that may facilitate determinations of cardiac wellness, and risk of sudden cardiac death, and may lead to clinical interventions to suppress hypertension such as medications and renal denervation procedures.

Optical sensor(s) 62 may be configured as a PPG sensor. Optical emitter(s) 63 may be configured to emit light signals belonging to a particular wavelength spectrum, and the optical detector(s) 64 may be configured to receive reflected light signals corresponding to the particular wavelength spectrum emitted by optical emitter(s) 63. In some examples, optical emitter(s) 63 may be configured to emit light signals belonging to a particular wavelength spectrum. Some examples of a particular wavelength spectrum may be an amber wavelength spectrum, a green wavelength spectrum, yellow wavelength spectrum, blue wavelength spectrum, or other wavelength spectrums. Optical detector(s) 64 may be configured to receive optical signals of the corresponding particular wavelength spectrum, such as an amber wavelength spectrum, a green wavelength spectrum, yellow wavelength spectrum, blue wavelength spectrum, or other wavelength spectrum.

Processing circuitry 50 may be configured to extract one or more features, such as cardiac features, from the optical signals. In some examples the features that may be extracted from the optical signals include one or more of peak-to-peak interval, systolic peak, time to systolic peak, systolic area, systolic slope trajectory, diastolic peak, diastolic area, diastolic slope trajectory, diastolic peak/systolic peak ratio, Boolean dicrotic notch, height of dicrotic notch, mean, standard deviation, kurtosis, and/or skewness. In some examples, processing circuitry 50 may be configured apply the extracted cardiac features to a machine learning model to determine the health condition status of the patient.

In some examples, processing circuitry 50 may be configured to extract one or more cardiac features from the optical signals, determine changes of the extracted cardiac features over the period of time, and apply one or more of the extracted cardiac features or the determined changes of the extracted cardiac features to a machine learning model to determine the health condition status of the patient.

In some examples, processing circuitry 50 may apply the optical signals and/or the levels or features described herein to a machine learning model to determine the health condition status of the patient. In some examples, processing circuitry 50 may be configured to identify a wavelength spectrum of the optical signals. In some examples, processing circuitry 50 may select a machine learning model is trained on training data having a wavelength spectrum that corresponds to the wavelength spectrum of the optical signals and apply the selected machine learning model, that corresponds to the wavelength spectrum of the optical signals, to the optical signals.

In some examples, IMD 10 may further include electrodes 16 and sensor(s) 61, other than optical sensor(s) 62, to monitor one or more physiological parameters of the patient, the one or more physiological parameters being different than blood pressure. In some examples, sensor(s) 61 may include an accelerometer. In some examples, physiological parameters may include one or more of heart rate, cardiac output, heart sounds, or impedance (e.g., fluid status).

Processing circuitry 50 may be further configured to determine the health condition status of patient 4 based on the determined blood pressure changes and the one or more physiological parameters of the patient. For example, processing circuitry 50 may apply the one or more physiological parameters to a machine learning model along with the optical signals to determine the health condition status of the patient. In some examples, processing circuitry 50 may determine and/or update a health condition status based on the one or more monitored physiological parameters and the health condition status determined based on changes in blood pressure.

In some examples, processing circuitry 50 may apply the optical signals and impedance measurements detected via electrodes 16, including one or more of real and reactive impedance, to the machine learning model to determine to determine the health condition status of the patient. In some examples, processing circuitry 50 may perform feature extraction on the optical signals and detected impedance measurements, including one or more of real and reactive impedance, and apply the extracted features to the machine learning model to determine to determine the health condition status of the patient.

FIG. 2B is a block diagram illustrating an example configuration of computing device 12. In some examples, computing device 12 takes the form of a smartphone, a laptop, a tablet computer, a personal digital assistant (PDA), a smartwatch or other wearable computing device. In some examples, computing devices 30 and/or server 24 may be configured similarly to the configuration of computing device 12 illustrated in FIG. 2B.

As shown in the example of FIG. 2B, computing device 12 may be logically divided into user space 202, kernel space 204, and hardware 206. Hardware 206 may include one or more hardware components that provide an operating environment for components executing in user space 202 and kernel space 204. User space 202 and kernel space 204 may represent different sections or segmentations of memory, where kernel space 204 provides higher privileges to processes and threads than user space 202. For instance, kernel space 204 may include operating system 220, which operates with higher privileges than components executing in user space 202.

As shown in FIG. 2B, hardware 206 includes processing circuitry 230, memory 232, one or more input devices 234, one or more output devices 236, one or more sensors 238, and communication circuitry 240. Although shown in FIG. 2B as a stand-alone device for purposes of example, computing device 12 may be any component or system that includes processing circuitry or other suitable computing environment for executing software instructions and, for example, need not necessarily include one or more elements shown in FIG. 2B.

Processing circuitry 230 is configured to implement functionality and/or process instructions for execution within computing device 12. For example, processing circuitry 230 may be configured to receive and process instructions stored in memory 232 that provide functionality of components included in kernel space 204 and user space 202 to perform one or more operations in accordance with techniques of this disclosure. Examples of processing circuitry 230 may include, any one or more microprocessors, controllers, GPUs, TPUs, DSPs, ASICs, FPGAs, or equivalent discrete or integrated logic circuitry.

Memory 232 may be configured to store information within computing device 12, for processing during operation of computing device 12. Memory 232, in some examples, is described as a computer-readable storage medium. In some examples, memory 232 includes a temporary memory or a volatile memory. Examples of volatile memories include RAM, DRAM, SRAM, and other forms of volatile memories known in the art. Memory 232, in some examples, also includes one or more memories configured for long-term storage of information, e.g. including non-volatile storage elements.

Examples of such non-volatile storage elements include magnetic hard discs, optical discs, floppy discs, flash memories, or forms of electrically programmable memories (EPROM) or electrically erasable and programmable (EEPROM) memories. In some examples, memory 232 includes cloud-associated storage.

One or more input devices 234 of computing device 12 may receive input, e.g., from patient 4, a clinician, or another user. Examples of input are tactile, audio, kinetic, and optical input. Input devices 234 may include, as examples, a mouse, keyboard, voice responsive system, camera, buttons, control pad, microphone, presence-sensitive or touch-sensitive component (e.g., screen), or any other device for detecting input from a user or a machine.

One or more output devices 236 of computing device 12 may generate output, e.g., to patient 4 or another user. Examples of output are tactile, haptic, audio, and visual output. Output devices 236 of computing device 12 may include a presence-sensitive screen, sound card, video graphics adapter card, speaker, cathode ray tube monitor, liquid crystal display (LCD), light emitting diodes (LEDs), or any type of device for generating tactile, audio, and/or visual output.

One or more sensors 238 of computing device 12 may sense physiological parameters or signals of patient 4. Sensor(s) 238 may include electrodes, accelerometers (e.g., 3-axis accelerometers), an optical sensor, impedance sensors, temperature sensors, pressure sensors, heart sound sensors (e.g., microphones or accelerometers), and other sensors, and sensing circuitry (e.g., including an ADC), similar to those described above with respect to IMDs 10 and FIG. 2A.

Communication circuitry 240 of computing device 12 may communicate with other devices by transmitting and receiving data. Communication circuitry 240 may receive patient parameter data from IMD 10, such as optical signal, from communication circuitry in IMD 10. Communication circuitry 240 may include a network interface card, such as an Ethernet card, an optical transceiver, a radio frequency transceiver, or any other type of device that can send and receive information. For example, communication circuitry 240 may include a radio transceiver configured for communication according to standards or protocols, such as 3G, 4G, 5G, WiFi (e.g., 802.11 or 802.15 ZigBee), Bluetooth®, or Bluetooth® Low Energy (BLE).

As shown in FIG. 2B, health monitoring application 250 executes in user space 202 of computing device 12. Health monitoring application 250 may be logically divided into presentation layer 252, application layer 254, and data layer 256. Presentation layer 252 may include a user interface (UI) component 260, which generates and renders user interfaces of health monitoring application 250.

Data layer 256 may include parameter data 290 and optical signal data 292, which may be received from IMD 10 via communication circuitry 240, and stored in memory 232 by processing circuitry 230. Application layer 254 may include, but is not limited to, status analyzer 270, and model configuration application 272. Status analyzer 270 may determine a health condition status based on optical signal data 292, and in some cases other parameter data 290, generated by IMD 10, as described herein. Status analyzer 270 may determine the health condition status based on application of the data as inputs one or more model(s) 294, which may include one or more machine learning models, algorithms, decision trees, and/or thresholds. In examples in which models 294 include one or more machine learning models, status analyzer 270 may apply feature vectors derived from the data to the model(s).

Model configuration component 272 may be configured to develop models 294 based on machine learning. Example machine learning techniques that may be employed to generate model(s) 294 can include various learning styles, such as supervised learning, unsupervised learning, and semi-supervised learning. Example types of algorithms include Bayesian algorithms, Markov models, Hawkes processes, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

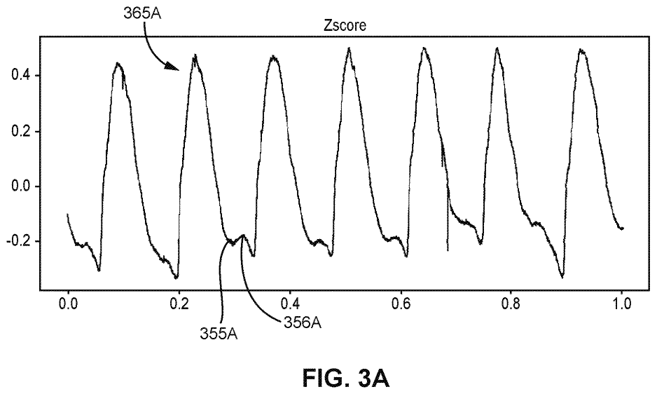

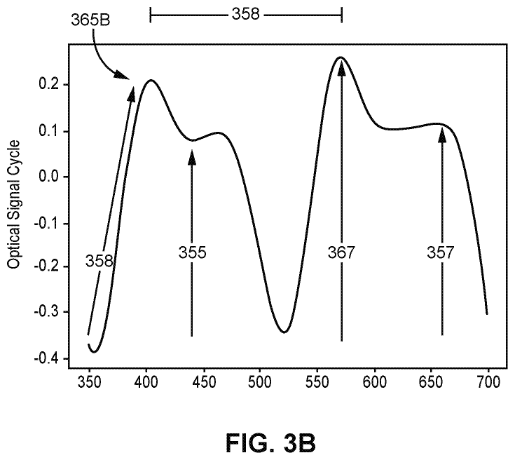

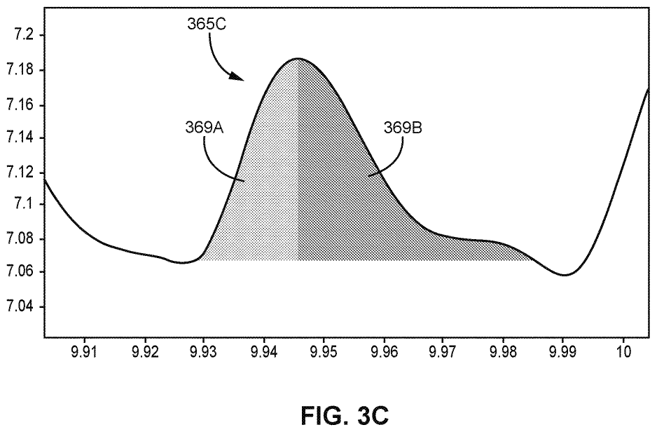

FIGS. 3A-3C show examples of optical signals 365A-365C (collectively, “optical signals 365” received by optical sensor(s) 62. Processing circuitry of system 2, e.g., processing circuitry 50, 23, and/or 230, may perform feature extraction to extract features from the optical signals. In some examples, the features that may be extracted from the optical signals include one or more of peak-to-peak interval 358, systolic peak 367, time to systolic peak 368, systolic area 369A, systolic slope trajectory, diastolic peak 357, diastolic area 369B, diastolic slope trajectory, diastolic peak/systolic peak ratio, Boolean dicrotic notch 355, height of dicrotic notch 355, mean, standard deviation, kurtosis, and/or skewness. As shown as an example in FIG. 3B, one or more features of time to systolic peak 368, peak-to-peak interval 358, dicrotic notch 355, diastolic peak 357, and/or systolic peak 367 are being extracted from the optical signal 365. As shown as an example in FIG. 3C, the features systolic area 369A and diastolic area 369B are being extracted from the optical signal 365. Processing circuitry of system 2, e.g., processing circuitry 50, 23, and/or 230, may apply the extracted features to a machine learning model to determine a health condition status. While FIGS. 3A-3C show an example of a dicrotic notch 355, diastolic peak 357, systolic peak 367, a dicrotic notch is a nadir point of a waveform, such as a pressure waveform, that indicates the end of systole. A dicrotic notch may occur after the closure of aortic valves and precedes the secondary dicrotic wave. A systole wave is a result of the direct pressure wave traveling from the left ventricle to the periphery of the body and a systolic peak is a maximum point of the systole wave. A diastolic wave is a result of reflection of the pressure wave by arteries of the lower body and a diastolic peak is a maximum point of the diastole wave.

In some examples, by extracting particular features from a received optical signal that have been determined according to the inventive techniques of this disclosure to correspond to blood pressure and/or particular health conditions related to blood pressure, as discussed above, to be applied to a machine learning model, the machine learning model may be more focused and spend a majority of its nodes on determining a blood pressure level, changes in blood pressure levels, which and/or a health condition status based on blood pressure changes over a period of time.

Processing circuitry of system 2 may be configured to execute an artificial intelligence (AI) engine that operates according to one or more models, such as machine learning models. Machine learning models may include any number of different types of machine learning models, such as neural networks, deep neural networks, convolution neural networks, recurrent neural networks, such as long short term memory networks, dense neural networks, and the like. In some examples, various feature inputs to the AI engine may be fed as direct inputs to different layers in a network and not necessarily prior to the convolution layers. Although described with respect to machine learning models, the techniques described in this disclosure are also applicable to other types of AI models, including rule-based models, finite state machines, and the like.

Machine learning may generally enable a computing device to analyze input data and identify an action to be performed responsive to the input data. Each machine learning model may be trained using training data that reflects likely input data. The training data may be labeled or unlabeled (meaning that the correct action to be taken based on a sample of training data is explicitly stated or not explicitly stated, respectively).

The training of the machine learning model may be guided (in that a designer, such as a computer programmer, may direct the training to guide the machine learning model to identify the correct action in view of the input data) or unguided (in that the machine learning model is not guided by a designer to identify the correct action in view of the input data). In some instances, the machine learning model is trained through a combination of labeled and unlabeled training data, a combination of guided and unguided training, or possibly combinations thereof. Examples of machine learning include nearest neighbor, naïve Bayes, decision trees, linear regression, support vector machines, neural networks, k-Means clustering, Q-learning, temporal difference, deep adversarial networks, evolutionary algorithms or other supervised, unsupervised, semi-supervised, or reinforcement learning algorithms to train one or more models.

Processing circuitry of system 2 may utilize machine learning, such as a deep learning algorithm or model (e.g., a neural network or deep belief network), to generate a score indicative of blood pressure level and/or changes in blood pressure level to determine a health condition status. Processing circuitry of system 2 may train a deep learning model to represent a relationship of the features extracted from the received optical signal discussed above to a blood pressure level, changes in blood pressure level, and/or a health condition status. For example, processing circuitry of system 2 may train the deep learning model using optical signals from other patients. In some examples, processing circuitry of system 2 may train the deep leaning model by adjusting the weights of a hidden layer of a neural network model to balance the contribution of each input (e.g., characteristics of the images input) according to determining a blood pressure level, changes in blood pressure level, and/or a health condition status. In some examples, processing circuitry of system 2 may train the deep learning model by also using optical signals received from patient 4 in addition to using optical signals from other patients.

Once the deep learning model is trained, processing circuitry of system may obtain and apply data, such as the features extracted from the received optical signal, to the trained deep learning model.

The output of the deep learning model may include a score that indicates a blood pressure level, changes in blood pressure level, and/or a health condition status. For example, the score may indicate a degree of hypertension of patient 4.

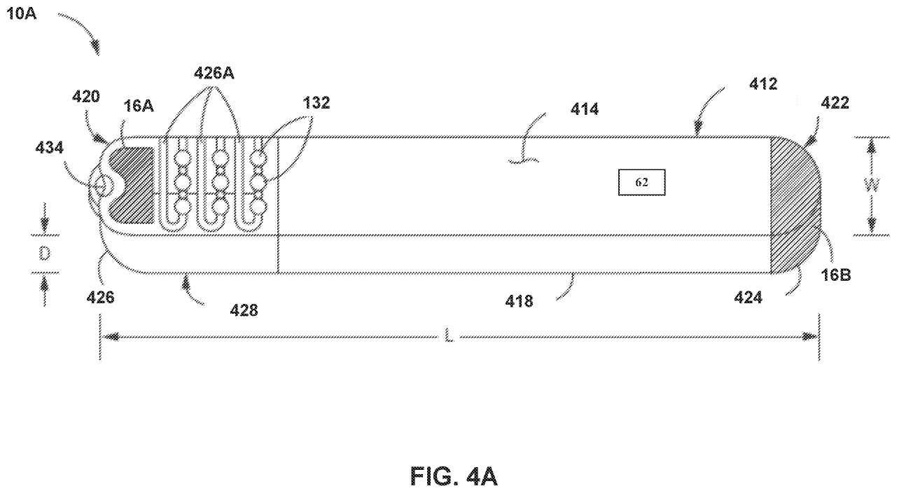

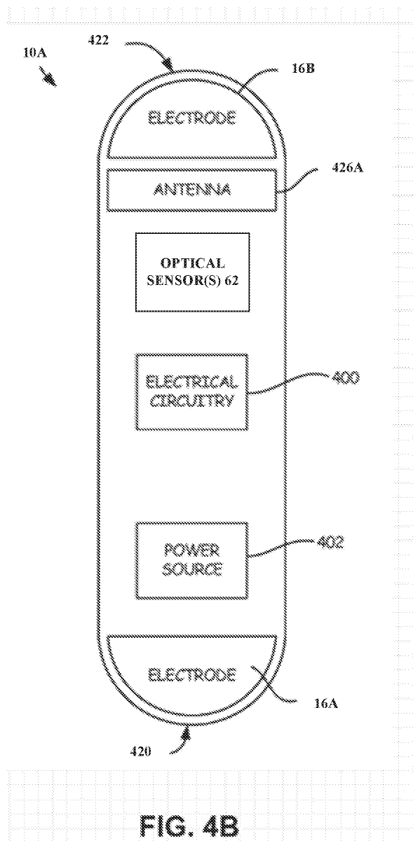

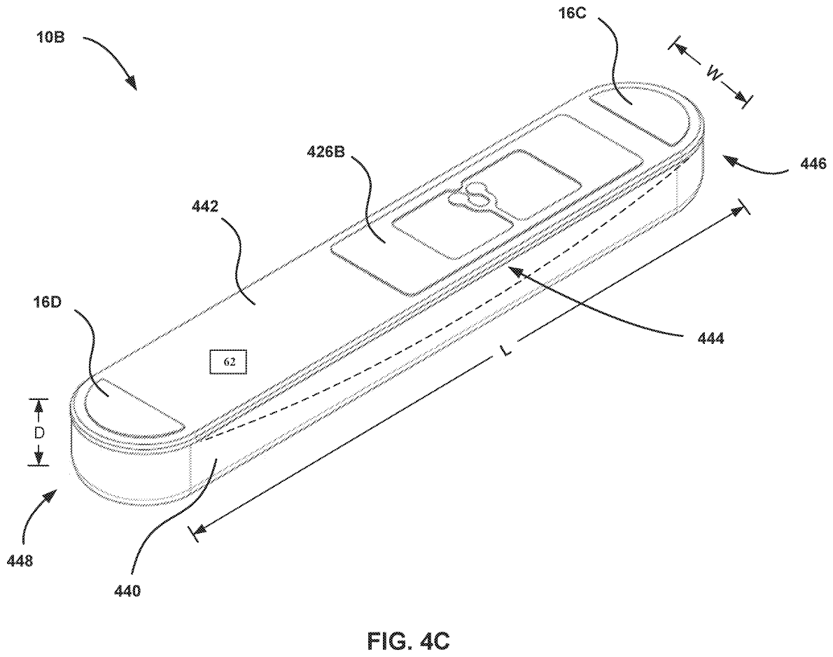

FIG. 4A is a conceptual drawing illustrating an IMD 10A, which may be an example configuration of IMD 10 of FIGS. 1A-2A as an implantable cardiac monitor (ICM). In the example shown in FIG. 4A, IMD 10A may be embodied as a monitoring device having housing 412, proximal electrode 16A, distal electrode 16B, and optical sensor(s) 62. The optical sensor(s) 62 may be positioned at various locations on IMD 10A. Housing 412 may further comprise first major surface 414, second major surface 418, proximal end 420, and distal end 422. Housing 412 encloses electronic circuitry located inside the IMD 10A and protects the circuitry contained therein from body fluids. Electrical feedthroughs provide electrical connection of electrodes 16A and 16B.

In the example shown in FIG. 4A, IMD 10A is defined by a length L, a width W and thickness or depth D and is in the form of an elongated rectangular prism wherein the length L is much larger than the width W, which in turn is larger than the depth D. In one example, the geometry of the IMD 10A—in particular a width W greater than the depth D—is selected to allow IMD 10A to be inserted under the skin of the patient using a minimally invasive procedure and to remain in the desired orientation during insertion.

For example, the device shown in FIG. 4A includes radial asymmetries (notably, the rectangular shape) along the longitudinal axis that maintains the device in the proper orientation following insertion. For example, the spacing between proximal electrode 16A and distal electrode 16B may range from 30 millimeters (mm) to 55 mm, 35 mm to 55 mm, and from 40 mm to 55 mm and may be any range or individual spacing from 25 mm to 60 mm. In addition, IMD 10A may have a length L that ranges from 30 mm to about 70 mm. In other examples, the length L may range from 5 mm to 60 mm, 15 mm to 50 mm, 40 mm to 60 mm, 45 mm to 60 mm and may be any length or range of lengths between about 5 mm and about 80 mm. In addition, the width W of major surface 414 may range from 5 mm to 15 mm, 3 mm to 10 mm, and may be any single or range of widths between 3 mm and 15 mm. The thickness of depth D of IMD 10A may range from 2 mm to 9 mm. In other examples, the depth D of IMD 10A may range from 2 mm to 5 mm, may range from 5 mm to 15 mm, and may be any single or range of depths from 2 mm to 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 4A, once inserted within the patient, the first major surface 414 faces outward, toward the skin of the patient while the second major surface 418 is located opposite the first major surface 414. In addition, in the example shown in FIG. 4A, proximal end 420 and distal end 422 are rounded to reduce discomfort and irritation to surrounding tissue once inserted under the skin of the patient. IMD 10A, including instrument and method for inserting IMDs 10 is described, for example, in U.S. Patent Publication No. 2014/0276928, incorporated herein by reference in its entirety.

Proximal electrode 16A and distal electrode 16B are used to sense cardiac signals, e.g. EGM signals, intra-thoracically or extra-thoracically, which may be sub-muscularly or subcutaneously. EGM signals may be stored in a memory of IMD 10A, and data may be transmitted via integrated antenna 426A to another medical device, which may be another implantable device or an external device, such as computing device 12. In some example, electrodes 16A and 16B may additionally or alternatively be used for sensing any bio-potential signal of interest, which may be, for example, an EGM, electroencephalogram (EEG), electromyogram (EMG), or a nerve signal, from any implanted location.

In the example shown in FIG. 4A, proximal electrode 16A is in close proximity to the proximal end 420 and distal electrode 16B is in close proximity to distal end 422. In this example, distal electrode 16B is not limited to a flattened, outward facing surface, but may extend from first major surface 414 around rounded edges 424 and/or end surface 425 and onto the second major surface 418 so that the electrode 16B has a three-dimensional curved configuration. In some examples, electrode 16B is an uninsulated portion of a metallic, e.g., titanium, part of housing 412.

In the example shown in FIG. 4A, proximal electrode 16A is located on first major surface 414 and is substantially flat, and outward facing. However, in other examples proximal electrode 16A may utilize the three dimensional curved configuration of distal electrode 16B, providing a three dimensional proximal electrode (not shown in this example). Similarly, in other examples distal electrode 16B may utilize a substantially flat, outward facing electrode located on first major surface 414 similar to that shown with respect to proximal electrode 16A.

The various electrode configurations allow for configurations in which proximal electrode 16A and distal electrode 16B are located on both first major surface 414 and second major surface 418. In other configurations, such as that shown in FIG. 4A, only one of proximal electrode 16A and distal electrode 16B is located on both major surfaces 414 and 418, and in still other configurations both proximal electrode 16A and distal electrode 16B are located on one of the first major surface 414 or the second major surface 418 (e.g., proximal electrode 16A located on first major surface 14 while distal electrode 16B is located on second major surface 418). In another example, IMD 10A may include electrodes on both major surface 414 and 418 at or near the proximal and distal ends of the device, such that a total of four electrodes are included on IMD 10A. Electrodes 16A and 16B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 4A, proximal end 420 includes a header assembly 428 that includes one or more of proximal electrode 16A, integrated antenna 426A, anti-migration projections 432, and/or suture hole 434. Integrated antenna 426A is located on the same major surface (i.e., first major surface 414) as proximal electrode 16A and is also included as part of header assembly 428. Integrated antenna 426A allows IMD 10A to transmit and/or receive data. In other examples, integrated antenna 426A may be formed on the opposite major surface as proximal electrode 16A, or may be incorporated within the housing 412 of IMD 10A. In the example shown in FIG. 4A, anti-migration projections 432 are located adjacent to integrated antenna 426A and protrude away from first major surface 414 to prevent longitudinal movement of the device. In the example shown in FIG. 4A, anti-migration projections 432 include a plurality (e.g., nine) small bumps or protrusions extending away from first major surface 414. As discussed above, in other examples anti-migration projections 432 may be located on the opposite major surface as proximal electrode 16A and/or integrated antenna 426A. In addition, in the example shown in FIG. 4A, header assembly 428 includes suture hole 434, which provides another means of securing IMD 10A to the patient to prevent movement following insertion. In the example shown, suture hole 434 is located adjacent to proximal electrode 16A. In one example, header assembly 428 is a molded header assembly made from a polymeric or plastic material, which may be integrated or separable from the main portion of IMD 10A.

FIG. 4B is a functional schematic diagram of IMD 10A as shown in FIG. 4A according to an embodiment of the present disclosure. IMD 10A may include proximal electrode 16B located at proximal end 422, distal electrode 16A located at distal end 420, optical sensor(s) 62, integrated antenna 426A, electrical circuitry 400 and power source 402. In particular, electrical circuitry 400 is coupled to proximal electrode 16B and distal electrode 16A to sense cardiac signals and monitor events. Electrical circuitry 400 may also connected to transmit and receive communications via integrated antenna 426A. Power source 402 provides power to electrical circuitry 400, as well as to any other components that require power. Power source 402 may include one or more energy storage devices, such as one or more rechargeable or non-rechargeable batteries. In some examples, electrical circuitry 400 includes processing circuitry 50 and storage device 56, such as a memory, as shown in FIG. 2, the memory 56 being operatively coupled to the processing circuitry 50 and configured to store a machine learning model.

In the example shown in FIG. 4B, electrical circuitry 400 may receive raw EGM signals monitored by proximal electrode 16B and distal electrode 16A and raw optical signals monitored by optical sensor(s) 62. Electrical circuitry 400 may include components/modules for converting the raw EGM signal to a processed EGM signal that can be analyzed to detect sense events and for converting the raw optical signals to a processed optical signal that can be analyzed to detect sense events. Although not shown, electrical circuitry 400 may include any discrete and/or integrated electronic circuit components that implement analog and/or digital circuits capable of producing the functions described for analyzing optical signals to determine a health condition status of a patient. For example, the electrical circuitry 400 may include analog circuits, e.g., pre-amplification circuits, filtering circuits, and/or other analog signal conditioning circuits. The modules may also include digital circuits, e.g., digital filters, combinational or sequential logic circuits, state machines, integrated circuits, a processor (shared, dedicated, or group) that executes one or more software or firmware programs, memory devices, or any other suitable components or combination thereof that provide the described functionality.

In one example, electrical circuitry 400 includes a sensing unit for monitoring the EGM signal detected by the respective proximal and distal electrodes 16A and 16B and light signals received by the optical sensor(s) 62, respectively. In one example, electrical circuitry 400 includes processing circuitry 50 that is utilized to receive information regarding sensed events and implements one or more algorithms for determining a health condition status of a patient. In addition, the analog voltage signals received from electrodes 16A and 16B may be passed to analog-to-digital (A/D) converters included in the electrical circuitry 400, and stored in a memory unit (not shown) included as part of electrical circuitry 400 for subsequent analysis with firmware executed by the processor included as part of electrical circuitry 400.

In some examples, housing 412 (FIG. 4A) may be a hermetically-sealed housing configured for subcutaneous implantation within the patient, wherein at least the power source 402, memory, and processing circuitry 50 are within the hermetically-sealed case.

FIG. 4C is a perspective drawing illustrating another IMD 10B, which may be another example configuration of IMD 10 from FIGS. 1A-2A. IMD 10B of FIG. 4C may be configured substantially similarly to IMD 10A of FIG. 4A, with differences between them discussed herein.

IMD 10B may include a leadless, subcutaneously-implantable monitoring device, e.g. an ICM. IMD 10B includes housing having a base 440 and an insulative cover 442. IMD 10B includes optical sensor(s) 62. Proximal electrode 16C and distal electrode 16D may be formed or placed on an outer surface of cover 442. Various circuitries and components of IMD 10B, e.g., described below with respect to FIG. 2A, may be formed or placed on an inner surface of cover 442, or within base 440. In some examples, a battery or other power source of IMD 10B may be included within base 440. In the illustrated example, antenna 426B is formed or placed on the outer surface of cover 442, but may be formed or placed on the inner surface in some examples. In some examples, insulative cover 442 may be positioned over an open base 440 such that base 440 and cover 442 enclose the circuitries and other components and protect them from fluids such as body fluids.

Circuitries and components may be formed on the inner side of insulative cover 442, such as by using flip-chip technology. Insulative cover 442 may be flipped onto a base 440. When flipped and placed onto base 440, the components of IMD 10B formed on the inner side of insulative cover 442 may be positioned in a gap 444 defined by base 440. Electrodes 16C and 16D and antenna 426B may be electrically connected to circuitry formed on the inner side of insulative cover 442 through one or more vias (not shown) formed through insulative cover 442. Insulative cover 442 may be formed of sapphire (i.e., corundum), glass, parylene, and/or any other suitable insulating material. Base 440 may be formed from titanium or any other suitable material (e.g., a biocompatible material). Electrodes 16C and 16D may be formed from any of stainless steel, titanium, platinum, iridium, or alloys thereof. In addition, electrodes 16C and 16D may be coated with a material such as titanium nitride or fractal titanium nitride, although other suitable materials and coatings for such electrodes may be used.

In the example shown in FIG. 4C, the housing of IMD 10B defines a length L, a width W and thickness or depth D and is in the form of an elongated rectangular prism wherein the length L is much larger than the width W, which in turn is larger than the depth D, similar to IMD 10A of FIG. 4C. For example, the spacing between proximal electrode 16C and distal electrode 16D may range from 30 millimeters (mm) to 50 mm, from 35 mm to 45 mm, or be approximately 40 mm. In addition, IMD 10B may have a length L that ranges from 30 mm to about 70 mm. In other examples, the length L may range from 5 mm to 60 mm, 40 mm to 60 mm, 45 mm to 55 mm, or be approximately 45 mm. In addition, the width W may range from 3 mm to 15 mm, such as approximately 8 mm. The thickness of depth D of IMD 10B may range from 2 mm to 15 mm, from 3 to 5 mm, or be approximately 4 mm. IMD 10B may have a volume of three cubic centimeters (cm) or less, or 1.5 cubic cm or less, such as approximately 1.4 cubic cm.

In the example shown in FIG. 4C, once inserted subcutaneously within the patient, outer surface of cover 442 faces outward, toward the skin of the patient. In addition, as shown in FIG. 4C, proximal end 446 and distal end 448 are rounded to reduce discomfort and irritation to surrounding tissue once inserted.

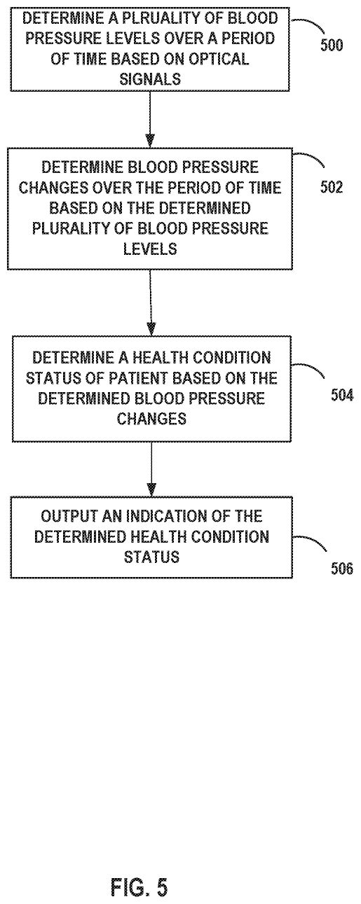

FIG. 5 is a flow diagram illustrating an example technique for operating system 2. As indicated by FIG. 5 processing circuitry may determine a plurality of blood pressure levels over a period of time based on optical signals (e.g., received reflected light signals) sensed by a subcutaneously-implanted medical device (e.g., an insertable cardiac monitor) (500). Processing circuitry 50 may determine blood pressure changes over the period of time based on the determined plurality of blood pressure levels (502). Processing circuitry 50 may determine a health condition status of patient 4 based on the determined blood pressure changes (504). Processing circuitry 50 may output an indication of the determined health condition status (506) to computing device 12 that may facilitate determinations of cardiac wellness, and risk of sudden cardiac death, and may lead to clinical interventions to suppress hypertension such as medications and renal denervation procedures.

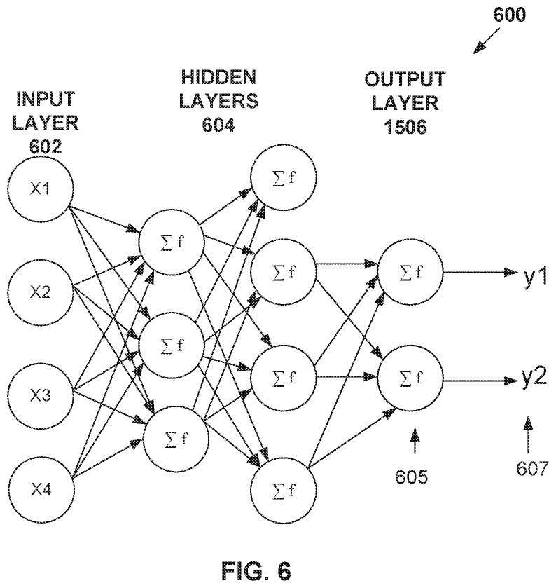

FIG. 6 is a conceptual diagram illustrating an example machine learning model 600 configured to determine a blood pressure level, changes in blood pressure level, and/or a health condition status. Machine learning model 600 is an example of the machine learning model discussed above. Machine learning model 600 is an example of a deep learning model, or deep learning algorithm, trained to determine a blood pressure level, changes in blood pressure level, and/or a health condition status. One or more of IMD 10, computing device 12, server 24, and/or other computing device(s) may train, store, and/or utilize machine learning model 600, but other devices may apply inputs associated with a particular patient to machine learning model 600 in other examples. As discussed above, other types of machine learning and deep learning models or algorithms may be utilized in other examples. For example, a convolutional neural network model of ResNet-18 may be used. Some non-limiting examples of models that may be used for transfer learning include AlexNet, VGGNet, GoogleNet, ResNet50, or DenseNet, etc. Some non-limiting examples of machine learning techniques include Support Vector Machines, K-Nearest Neighbor algorithm, and Multi-layer Perceptron.

As shown in the example of FIG. 6, machine learning model 600 may include three layers. These three layers include input layer 602, hidden layer 604, and output layer 606. Output layer 606 comprises the output from the transfer function 605 of output layer 606. Input layer 602 represents each of the input values X1 through X4 provided to machine learning model 600. In some examples, the input values may be any of the of values input into the machine learning model, as described above. For example, the input values may be one of more features extracted from the received optical signal, as described above. In some examples, the input values may be one or more of peak-to-peak interval, systolic peak, time to systolic peak, systolic area, systolic slope trajectory, diastolic peak, diastolic area, diastolic slope trajectory, diastolic peak/systolic peak ratio, Boolean dicrotic notch, height of dicrotic notch, mean, standard deviation, kurtosis, and/or skewness. In addition, in some examples input values of machine learning model 1500 may include additional data, such as data relating to one or more additional parameters of patient 4.

Each of the input values for each node in the input layer 602 is provided to each node of hidden layer 604. In the example of FIG. 6, hidden layers 604 include two layers, one layer having four nodes and the other layer having three nodes, but fewer or greater number of nodes may be used in other examples. Each input from input layer 602 is multiplied by a weight and then summed at each node of hidden layers 604. During training of machine learning model 600, the weights for each input are adjusted to establish the relationship between the extracted cardiac features to determining a blood pressure level, changes in blood pressure level, and/or a health condition status. In some examples, one hidden layer may be incorporated into machine learning model 600, or three or more hidden layers may be incorporated into machine learning model 600, where each layer includes the same or different number of nodes.

The result of each node within hidden layers 604 is applied to the transfer function of output layer 606. The transfer function may be liner or non-linear, depending on the number of layers within machine learning model 600. Example non-linear transfer functions may be a sigmoid function or a rectifier function. The output 607 of the transfer function may be a classification of a blood pressure level, changes in blood pressure level, and/or a score indicative of a health condition status of a patient, such as a degree of hypertension, that is generated by a computing device or computing system, such as by processing circuitry 50, in response to applying the extracted cardiac features from the received optical signal to machine learning model 600.

As shown in the example above, by extracting one or more particular cardiac features from the received optical signal, and applying the extracted cardiac features to a machine learning model, such as machine learning model 600, to determine a blood pressure level, changes in blood pressure level, and/or a score indicative of a health condition status of a patient, processing circuitry 50 is able to determine a degree of hypertension of a patient with great accuracy, specificity, and sensitivity. This may facilitate determinations of cardiac wellness, and risk of sudden cardiac death, and may lead to clinical interventions to suppress hypertension such as medications and renal denervation procedures.

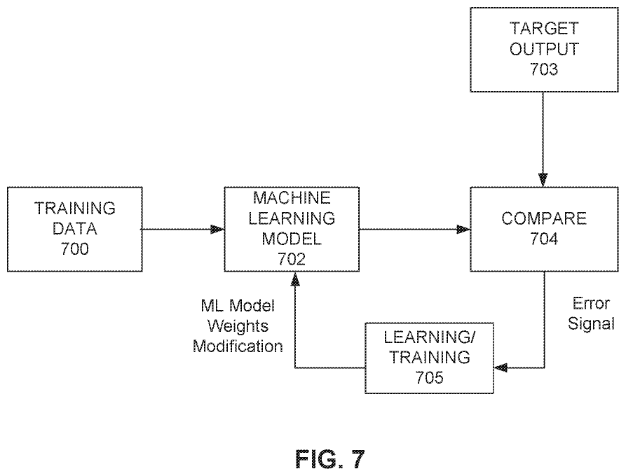

FIG. 7 is an example of the machine learning model 702 being trained using supervised and/or reinforcement learning techniques. The machine learning model 702 may be implemented using any number of models for supervised and/or reinforcement learning, such as but not limited to, an artificial neural network, a decision tree, naïve Bayes network, support vector machine, or k-nearest neighbor model, to name only a few examples. In some examples, one or more of IMD 10, computing device 12, server 24, and/or other computing device(s) initially trains the machine learning model 702 based on a training set of metrics and corresponding to a received optical signal. The training set 700 may include a set of feature vectors, where each feature in the feature vector represents a value for a particular metric. One or more of IMD 10, computing device 12, server 24, and/or other computing device(s) may select a training set comprising a set of training instances, each training instance comprising an association between one or more respective optical signal features of a respective cardiac feature and a respective optical signal. A prediction or classification by the machine learning model 702 may be compared 704 to the target output 703. Based on an error signal representing the comparison, the processing circuitry implementing a learning/training function 705 may send or apply a modification to weights of machine learning model 702 or otherwise modify/update the machine learning model 702. For example, one or more of IMD 10, computing device 12, server 24, and/or other computing device(s) may, for each training instance in the training set, modify, based on the respective cardiac features and the respective optical signal of the training instance, the machine learning model 702 to change a score generated by the machine learning model 702 in response to subsequent optical signals applied to the machine learning model 702.

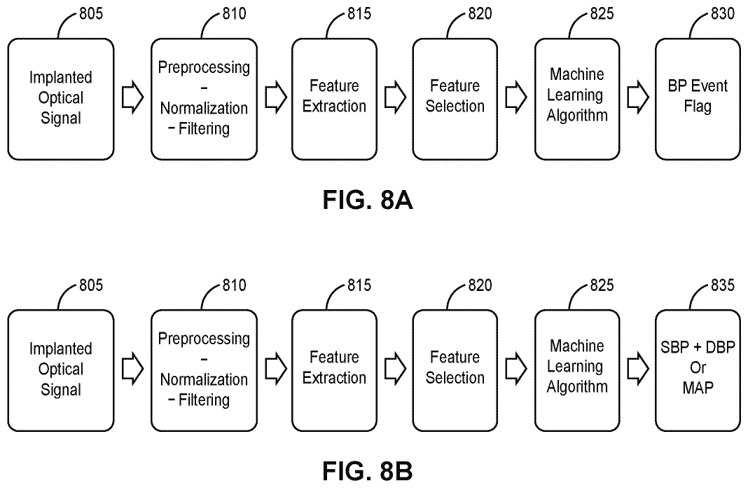

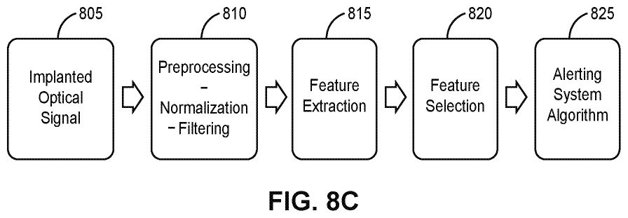

FIGS. 8A-8C show different examples of flow diagrams showing of processing circuitry 50 determining a health condition status based on optical signals received by optical sensor(s) 62. For example, processing circuitry 50 may the pre-process, normalize, and/or filter 810 the optical signals 805. Processing circuitry 50 may further extract features 815, as described above. Processing circuitry 50 may select extracted features 820, as described above. As shown in FIGS. 8A-8B, processing circuitry 50 may input the selected features to a machine learning model 825. As shown as an example in FIG. 8A, processing circuitry 50 may generate an output of a blood pressure event flag 830, such as classifying the blood pressure that indicates a health condition status. Processing circuitry 50 may determine whether to generate a health alert or not generate a health alert based on the classification and/or the health condition status. As shown as an example in FIG. 8B, processing circuitry 50 may generate an output of a value of blood pressure wave, such as diastolic blood pressure, systolic blood pressure, and/or mean blood pressure 835. Processing circuitry 50 may determine a health condition status based on the determined blood pressure values. Processing circuitry 50 may determine whether to generate a health alert based on the determined health condition status and/or the determined blood pressure values. As shown in FIG. 8C, processing circuitry 50 may determine a health condition status based on the selected features and determine whether to generate a health alert based on the determined health condition.

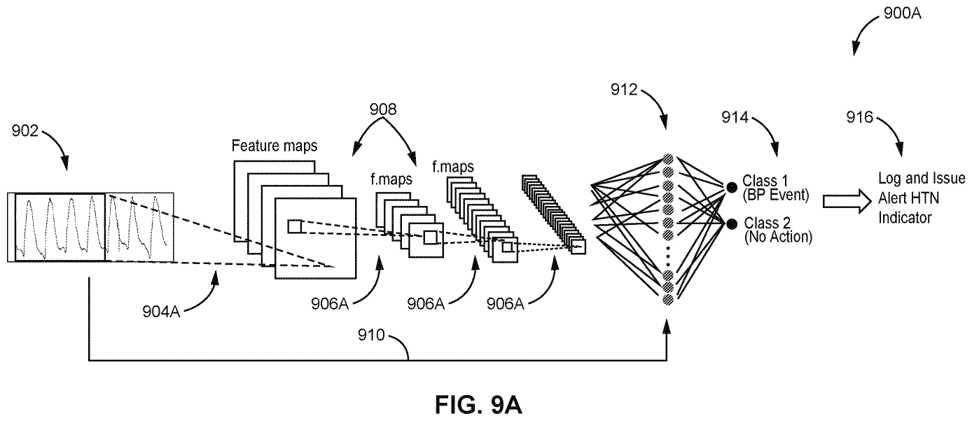

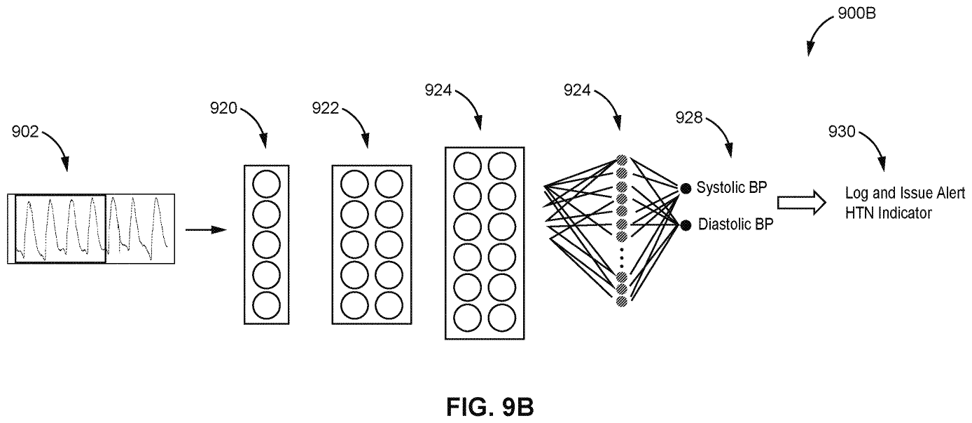

FIGS. 9A-9B show different examples of processing circuitry of system 2, e.g., processing circuitry 50, 23, and/or 230, determining a health condition status based on optical signals received by optical sensor(s) 62 and/or selected features from the optical signals, as discussed above. For example, a machine learning model 900A, as shown in FIG. 9A, may be a blood pressure event classification-based convolutional neural network. As shown in FIG. 9A, input 902 may be optical signals received by optical sensor(s) 62.

The processing circuitry may perform one or more convolution actions 904A and 904B (collectively, “convolutions 904”) and one or subsampling actions 906A and 906B (collectively, “subsamplings 906”) on input data 902 to determine feature maps 908 which may be input for classifier 912, e.g., with additional features 910 derived from input data 902. For example, as shown in the example in FIG. 9A, the health condition status may be an indication of a classification blood pressure event 914. For example, optical signals being classified as class 1 may indicate a health condition status that indicates to generate a health alert 916. Optical signals being classified as class 2 may indicate a health condition status that indicates a health alert does not need to be generated.

In some examples, a machine learning model, as shown in FIG. 9B, may be a blood pressure regression based CNN 900B. As shown in FIG. 9B, input 902 may be optical signals received by optical sensor(s) 62. Processing circuitry of system 2 may perform a sequence of operations on the input optical signal samples 902. In the example of FIG. 9B, processing circuitry of system 2 may generate an input sequence 920 based on the optical signal samples, and apply input sequent to a CNN 922. Output of CNN 922 may be applied to an LSTM 924. Processing circuitry of system 2 applies output of LSTM 924 to one or more fully connected layers 926 that generate a regression output of model 900B. For example, as shown in the example in FIG. 9B, the health condition status may be an indication of a blood pressure value, such as systolic blood pressure or diastolic blood pressure. Processing circuitry may determine a health condition status based on the determined blood pressure values. Processing circuitry may determine whether to generate a health alert 930 based on the determined health condition status and/or the determined blood pressure values.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

The following examples are illustrative of the techniques described herein.

Example 1: An implantable medical device includes a housing configured for subcutaneous implantation at a location within a patient; an optical sensor, the optical sensor being at least one of on or within the housing, the optical sensor configured to generate optical signals indicative of blood movement in vessels of the patient proximate to the location over a period of time; and processing circuitry configured to: determine a plurality of blood pressure levels over the period of time based on the optical signals; determine blood pressure changes over the period of time based on the determined plurality of blood pressure levels; determine a health condition status of the patient based on the determined blood pressure changes over the period of time; and output an indication of the determined health condition status.

Example 2: The implantable device of example 1, wherein the optical sensor is a photoplethysmography sensor.

Example 3: The implantable device of any of examples 1-2, wherein the optical sensor is configured to emit light signals belonging to a particular wavelength spectrum, and the optical sensor is configured to receive reflected light signals of the particular wavelength spectrum.

Example 4: The implantable device of example 3, wherein the particular wavelength spectrum is amber wavelength spectrum or green wavelength spectrum.

Example 5: The implantable device of any of examples 1-4, wherein to determine a health condition status of the patient based on the determined blood pressure changes over the period of time, the processing circuitry is further configured to: extract one or more cardiac features from the optical signals; and apply the extracted cardiac features to a machine learning model to determine the health condition status of the patient.

Example 6: The implantable device of any of examples 1-4, wherein to determine a health condition status of the patient based on the determined blood pressure changes over the period of time, the processing circuitry is further configured to: extract one or more cardiac features from the optical signals; determine changes of the extracted cardiac features over the period of time; and apply one or more of the extracted cardiac features or the determined changes of the extracted cardiac features to a machine learning model to determine the health condition status of the patient.

Example 7: The implantable device of any of examples 1-4, wherein the processing circuitry is further configured to: apply the optical signals to a machine learning model to determine the health condition status of the patient.

Example 8: The implantable device of any of examples 5-7, wherein the machine learning model is trained on training data having a wavelength spectrum that corresponds to the wavelength spectrum of the optical signals.

Example 9: The implantable device of any of examples 1-8, wherein the processing circuitry is further configured to identify a wavelength spectrum of the received light signals.

Example 10: The implantable device of any of examples 1-9, further includes one or more second sensors to monitor one or more physiological parameters of the patient, the one or more physiological parameters being different than blood pressure, wherein the processing circuitry is further configured to determine the health condition status of the patient based on the determined blood pressure changes and the one or more physiological parameters of the patient.

Example 11: The implantable device of example 10, wherein the one or more second sensors comprise at least one of an accelerometer or an electrode.

Example 12: The implantable device of any of examples 10-11, wherein the one or more physiological parameters comprise at least one of heart rate, cardiac output, heart sounds, or impedance.

Example 13: The implantable device of any of examples 1-12, wherein the implantable device is an insertable cardiac monitor, the insertable cardiac monitor includes a power source operatively coupled to the processing circuitry; a memory operatively coupled to the processing circuitry and configured to store the machine learning model; a distal electrode operatively coupled to the processing circuitry; a proximal electrode operatively coupled to the processing circuitry; and a hermetically-sealed housing configured for subcutaneous implantation within the patient, wherein at least the power source, memory, and processing circuitry are within the hermetically-sealed case, and wherein the housing has a length, a width, and a depth, wherein the length is greater than the width and the width is greater than the depth, wherein the length is within a range from 5 millimeters (mm) to 60 mm, wherein the width is within a range from 5 mm to 15 mm, and wherein the depth is within a range from 5 mm to 15 mm.

Example 14: The implantable device of any of examples 1-13, wherein the health condition status is a degree of hypertension.

Example 15: A system includes an implantable medical device (IMD) includes a housing configured for subcutaneous implantation at a location within a patient; and an optical sensor, the optical sensor being at least one of on or within the housing, the optical sensor configured to generate optical signals indicative of blood movement in vessels of the patient proximate to the location over a period of time; and one or more computing devices communicatively coupled to the IMD, wherein the one or more computing devices comprise: a memory; and processing circuitry coupled to the memory, the processing circuitry being configured to: determine a plurality of blood pressure levels over the period of time based on the optical signals; determine blood pressure changes over the period of time based on the determined plurality of blood pressure levels; determine a health condition status of the patient based on the determined blood pressure changes over the period of time; and output an indication of the determined health condition status.

Example 16: The system of example 15, wherein the optical sensor is a photoplethysmography sensor.

Example 17: The system of any of examples 15-16, wherein the optical sensor is configured to emit light signals belonging to a particular wavelength spectrum, and the optical sensor is configured to receive reflected light signals of the particular wavelength spectrum.

Example 18: The system of example 17, wherein the particular wavelength spectrum is amber wavelength spectrum or green wavelength spectrum.

Example 19: The system of any of examples 15-18, wherein to determine a health condition status of the patient based on the determined blood pressure changes over the period of time, the processing circuitry is further configured to: extract one or more cardiac features from the optical signals; and apply the extracted cardiac features to a machine learning model to determine the health condition status of the patient.

Example 20: The system of any of examples 15-18, wherein to determine a health condition status of the patient based on the determined blood pressure changes over the period of time, the processing circuitry is further configured to: extract one or more cardiac features from the optical signals; determine changes of the extracted cardiac features over the period of time; and apply one or more of the extracted cardiac features or the determined changes of the extracted cardiac features to a machine learning model to determine the health condition status of the patient.

Example 21: The system of any of examples 15-18, wherein the processing circuitry is further configured to: apply the optical signals to a machine learning model to determine the health condition status of the patient.

Example 22: The system of any of examples 19-21, wherein the machine learning model is trained on training data having a wavelength spectrum that corresponds to the wavelength spectrum of the optical signals.

Example 23: The system of any of examples 15-22, wherein the processing circuitry is further configured to identify a wavelength spectrum of the optical signals.

Example 24: The system of any of examples 15-23, the implantable medical device further includes one or more second sensors to monitor one or more physiological parameters of the patient, the one or more physiological parameters being different than blood pressure, and wherein the processing circuitry is further configured to determine the health condition status of the patient based on the determined blood pressure changes and the one or more physiological parameters of the patient.

Example 25: The system of example 24, wherein the one or more second sensors include at least one of an accelerometer or an electrode.

Example 26: The system of any of examples 23-24, wherein the one or more physiological parameters are at least one of heart rate, cardiac output, heart sounds, or impedance.

Example 27: The system of any of examples 15-26, wherein the implantable device is an insertable cardiac monitor, the insertable cardiac monitor includes a power source operatively coupled to insertable cardiac monitor processing circuitry; a memory operatively coupled to the insertable cardiac monitor processing circuitry and configured to store the machine learning model; a distal electrode operatively coupled to the insertable cardiac monitor processing circuitry; a proximal electrode operatively coupled to the insertable cardiac monitor processing circuitry; and a hermetically-sealed housing configured for subcutaneous implantation within the patient, wherein at least the power source, memory, and insertable cardiac monitor processing circuitry are within the hermetically-sealed case, and wherein the housing has a length, a width, and a depth, wherein the length is greater than the width and the width is greater than the depth, wherein the length is within a range from 5 millimeters (mm) to 60 mm, wherein the width is within a range from 5 mm to 15 mm, and wherein the depth is within a range from 5 mm to 15 mm.

Example 28: The system of any of examples 15-27, wherein the health condition status is a degree of hypertension.

Example 29: A method for operating processing circuitry of a medical system includes receiving, by the processing circuitry, optical signals indicative of blood movement in vessels of a patient via an implantable medical device, including an optical sensor, implanted subcutaneously within the patient; determining, by the processing circuitry, a plurality of blood pressure levels of the patient over a period of time based on the optical signals; determining, by the processing circuitry, blood pressure changes over the period of time based on the determined plurality of blood pressure levels; determining, by the processing circuitry, a health condition status of the patient based on the determined blood pressure changes over the period of time; and outputting, by the processing circuitry, an indication of the determined health condition status.

Example 30: The method of example 29, wherein the optical sensor is a photoplethysmography sensor.

Example 31: The method of any of examples 29-30, wherein the optical signals belong to a particular wavelength spectrum.

Example 32: The method of example 31, wherein the particular wavelength spectrum is amber wavelength spectrum or green wavelength spectrum.

Example 33: The method of any of examples 29-32, wherein to determine a health condition status of the patient based on the determined blood pressure changes over the period of time, the method further comprises: extracting, by the processing circuitry, one or more cardiac features from the optical signals; and applying, by the processing circuitry, the extracted cardiac features to a machine learning model to determine the health condition status of the patient.

Example 34: The method of any of examples 29-32, wherein to determine a health condition status of the patient based on the determined blood pressure changes over the period of time, the method further comprises: extracting, by the processing circuitry, one or more cardiac features from the optical signals; determining, by the processing circuitry, changes of the extracted cardiac features over the period of time; and applying, by the processing circuitry, one or more of the extracted cardiac features or the determined changes of the extracted cardiac features to a machine learning model to determine the health condition status of the patient.

Example 35: The method of any of examples 29-32, wherein the method further comprises: applying, by the processing circuitry, the optical signals to a machine learning model to determine the health condition status of the patient.

Example 36: The method of any of examples 29-35, wherein the machine learning model is trained on training data having a wavelength spectrum that corresponds to the wavelength spectrum of the optical signals.

Example 37: The method of any of examples 29-36, wherein the method further comprises identifying a wavelength spectrum of the optical signals.

Example 38: The method of any of examples 29-36, the method further includes receiving, by the processing circuitry, one or more physiological parameters of the patient via one or more second sensors, the one or more physiological parameters being different than blood pressure; and determining, by the processing circuitry, the health condition status of the patient based on the determined blood pressure changes and the one or more physiological parameters of the patient.

Example 39: The method of example 38, wherein the one or more second sensors include at least one of an accelerometer or an electrode.

Example 40: The method of any of examples 38-39, wherein the one or more physiological parameters are at least one of heart rate, cardiac output, heart sounds, or impedance.

Example 41: The method of any of examples 29-40, wherein the health condition status is a degree of hypertension.

Example 42: A non-transitory computer-readable storage medium having stored thereon instructions that, when executed, cause one or more processors to at least: receive optical signals indicative of blood movement in vessels of a patient via an implantable medical device, including an optical sensor, implanted subcutaneously within the patient; determine a plurality of blood pressure levels of the patient over a period of time based on the optical signals; determine blood pressure changes over the period of time based on the determined plurality of blood pressure levels; determine a health condition status of the patient based on the determined blood pressure changes over the period of time; and output an indication of the determined health condition status.

Various examples have been described. These and other examples are within the scope of the following claims.

1. An implantable medical device comprising:
a housing configured for subcutaneous implantation at a location within a patient;
an optical sensor, the optical sensor being at least one of on or within the housing, the optical sensor configured to generate optical signals indicative of blood movement in vessels of the patient proximate to the location over a period of time; and
processing circuitry configured to:
determine a plurality of blood pressure levels over the period of time based on the optical signals;
determine blood pressure changes over the period of time based on the determined plurality of blood pressure levels;
determine a health condition status of the patient based on the determined blood pressure changes over the period of time; and
output an indication of the determined health condition status.

2. The implantable device of claim 1, wherein the optical sensor is configured to emit light signals belonging to a particular wavelength spectrum, and
the optical sensor is configured to receive reflected light signals of the particular wavelength spectrum.

3. The implantable device of claim 1, wherein to determine a health condition status of the patient based on the determined blood pressure changes over the period of time, the processing circuitry is further configured to:
extract one or more cardiac features from the optical signals; and
apply the extracted cardiac features to a machine learning model to determine the health condition status of the patient.

4. The implantable device of claim 1, wherein to determine a health condition status of the patient based on the determined blood pressure changes over the period of time, the processing circuitry is further configured to:
extract one or more cardiac features from the optical signals;
determine changes of the extracted cardiac features over the period of time; and
apply one or more of the extracted cardiac features or the determined changes of the extracted cardiac features to a machine learning model to determine the health condition status of the patient.

5. The implantable device of claim 1, wherein the processing circuitry is further configured to:
apply the optical signals to a machine learning model to determine the health condition status of the patient.

6. The implantable device of claim 3, wherein the machine learning model is trained on training data having a wavelength spectrum that corresponds to the wavelength spectrum of the optical signals.

7. The implantable device of claim 1, wherein the processing circuitry is further configured to identify a wavelength spectrum of the received light signals.

8. The implantable device of claim 1, further comprising:
one or more second sensors to monitor one or more physiological parameters of the patient, the one or more physiological parameters being different than blood pressure,
wherein the processing circuitry is further configured to determine the health condition status of the patient based on the determined blood pressure changes and the one or more physiological parameters of the patient.

9. The implantable device of claim 1, wherein the implantable device is an insertable cardiac monitor, the insertable cardiac monitor comprising:
a power source operatively coupled to the processing circuitry;
a memory operatively coupled to the processing circuitry and configured to store the machine learning model;
a distal electrode operatively coupled to the processing circuitry;
a proximal electrode operatively coupled to the processing circuitry; and
a hermetically-sealed housing configured for subcutaneous implantation within the patient, wherein at least the power source, memory, and processing circuitry are within the hermetically-sealed case, and
wherein the housing has a length, a width, and a depth,
wherein the length is greater than the width and the width is greater than the depth,
wherein the length is within a range from 5 millimeters (mm) to 60 mm,
wherein the width is within a range from 5 mm to 15 mm, and
wherein the depth is within a range from 5 mm to 15 mm.

10. A system comprising:
an implantable medical device (IMD) comprising:
a housing configured for subcutaneous implantation at a location within a patient; and
an optical sensor, the optical sensor being at least one of on or within the housing, the optical sensor configured to generate optical signals indicative of blood movement in vessels of the patient proximate to the location over a period of time; and
one or more computing devices communicatively coupled to the IMD, wherein the one or more computing devices comprise:
a memory; and
processing circuitry coupled to the memory, the processing circuitry being configured to:
determine a plurality of blood pressure levels over the period of time based on the optical signals;
determine blood pressure changes over the period of time based on the determined plurality of blood pressure levels;
determine a health condition status of the patient based on the determined blood pressure changes over the period of time; and
output an indication of the determined health condition status.

11. The system of claim 10, wherein the optical sensor is configured to emit light signals belonging to a particular wavelength spectrum, and
the optical sensor is configured to receive reflected light signals of the particular wavelength spectrum.

12. The system of claim 10, wherein to determine a health condition status of the patient based on the determined blood pressure changes over the period of time, the processing circuitry is further configured to:
extract one or more cardiac features from the optical signals; and
apply the extracted cardiac features to a machine learning model to determine the health condition status of the patient.

13. The system of claim 10, wherein to determine a health condition status of the patient based on the determined blood pressure changes over the period of time, the processing circuitry is further configured to:
extract one or more cardiac features from the optical signals;
determine changes of the extracted cardiac features over the period of time; and
apply one or more of the extracted cardiac features or the determined changes of the extracted cardiac features to a machine learning model to determine the health condition status of the patient.

14. The system of claim 10, wherein the processing circuitry is further configured to:
apply the optical signals to a machine learning model to determine the health condition status of the patient.

15. The system of claim 12, wherein the machine learning model is trained on training data having a wavelength spectrum that corresponds to the wavelength spectrum of the optical signals.

16. A method for operating processing circuitry of a medical system comprising:
receiving, by the processing circuitry, optical signals indicative of blood movement in vessels of a patient via an implantable medical device, including an optical sensor, implanted subcutaneously within the patient;
determining, by the processing circuitry, a plurality of blood pressure levels of the patient over a period of time based on the optical signals;
determining, by the processing circuitry, blood pressure changes over the period of time based on the determined plurality of blood pressure levels;
determining, by the processing circuitry, a health condition status of the patient based on the determined blood pressure changes over the period of time; and
outputting, by the processing circuitry, an indication of the determined health condition status.

17. The method of claim 16, wherein to determine a health condition status of the patient based on the determined blood pressure changes over the period of time, the method further comprises:
extracting, by the processing circuitry, one or more cardiac features from the optical signals; and
applying, by the processing circuitry, the extracted cardiac features to a machine learning model to determine the health condition status of the patient.

18. The method of claim 16, wherein to determine a health condition status of the patient based on the determined blood pressure changes over the period of time, the method further comprises:
extracting, by the processing circuitry, one or more cardiac features from the optical signals;
determining, by the processing circuitry, changes of the extracted cardiac features over the period of time; and
applying, by the processing circuitry, one or more of the extracted cardiac features or the determined changes of the extracted cardiac features to a machine learning model to determine the health condition status of the patient.

19. The method of claim 16, wherein the method further comprises:
applying, by the processing circuitry, the optical signals to a machine learning model to determine the health condition status of the patient.

20. The method of claim 16, the method further comprising:
receiving, by the processing circuitry, one or more physiological parameters of the patient via one or more second sensors, the one or more physiological parameters being different than blood pressure; and
determining, by the processing circuitry, the health condition status of the patient based on the determined blood pressure changes and the one or more physiological parameters of the patient.
